(19) United States
(12) Patent Application Publication
(10) Pub. No.: US 2023/0041820 A1
(43) Pub. Date: Feb. 9, 2023

(54) DETECTION OF PLASTIC MICROPARTICLES BY FLOW CYTOMETRY

(71) Applicant: SOCIETE DES PRODUITS NESTLE S.A., Vevey (CH)

(72) Inventors: FILIPPO DE FRANCESCHI, Lussery-Villars (CH); RAFAEL PINTO PEIXOTO, Lausanne (CH); ORNELLA COMINETTI ALLENDE, Denges (CH); ALESSIO G. PALINI, I-Cambiago, MI (IT)

(21) Appl. No.: 17/758,808

(22) PCT Filed: Jan. 14, 2021
(86) PCT No.: PCT/EP2021/050610

§ 371 Date: Jul. 14, 2022

(30) Foreign Application Priority Data
Jan. 15, 2020 (EP) 20151953.5

(51) Int. Cl. G01N 15/14; G01N 33/44

(52) U.S. Cl. CPC G01N 15/1434; G01N 15/1404; G01N 33/442; G01N 2015/1006

(57) ABSTRACT

The present invention relates generally to the field of plastic microparticles. In particular, the present invention relates to the detection of plastic microparticles in a water-based sample. An embodiment of the present invention relates to a process for detecting and characterizing plastic microparticles in a water-based sample comprising the analysis of the sample by spectral flow cytometry. In accordance with the present invention, the process described herein may comprise the processing of the recorded flow cytometry data by a machine learning algorithm that can distinguish and categorize each particle based on its unique spectrum to characterize, for example, the plastic microparticles.

The present invention relates generally to the field of plastic microparticles. In particular, the present invention relates to the detection of plastic microparticles in a water-based sample. An embodiment of the present invention relates to a process for detecting plastic microparticles in a water-based sample comprising the analysis of the sample by flow cytometry. In accordance with the present invention, the process described herein may comprise the processing of the recorded flow cytometry data by a machine learning algorithm that can distinguish and categorize each particle based on its unique spectrum to characterize, for example, the plastic microparticles.

Plastic microparticles, sometimes referred to as microplastics, are× generated from and used in several consumer products, for example some cosmetic products, and may furthermore result from the degradation of larger objects. Recently, microplastics have been found in the air, in seawater, in sediments and even in tissues of some animals. Microplastics have been recently described as persistent, pervasive environmental pollutants which may have effects on nutritional state, histology, enzyme function, and life span of some species [Waste (Second Edition), A Handbook for Management, 2019, Pages 405-424].

Hence, the importance of the detection of plastic microparticles, which are plastic particles with a size in the range of 1 um to 5 mm, is pivotal for environmental studies, pollution detection and monitoring and food safety.

Several methods are used in the art to detect microplastics in water-based samples. Such methods are, for example reviewed Trends in Analytical Chemistry 110 (2019) 150 - 159, hereby incorporated by reference in its entirety. The current gold standard technologies to identify microplastics are Raman spectroscopy and FTIR spectroscopy.

With micro-Raman, it is possible to identify particles as small as 1 µm, while with micro-FTIR the lowest size limit is currently about 20 µm.

Other methods of detection of microplastics in water have been developed and include the staining of the plastic particles with Nile Red dissolved in acetone or methanol or other organic solvents to allow the dye to bind to the plastic. Counting of microplastics involves sample filtration and imaging of the filter under mercury lamp or laser illumination.

However, the current state-of-the-art methods for microplastics detection have several disadvantages. In general, spectroscopic methods are relatively slow. Such methods are somewhat subjective as they rely on the identification of plastic particles by visual inspection via microscopy. Further, staining, such as with Nile Red for example, is known to occasionally create misleading artifacts and does not differentiate between the different types of plastic.

It would therefore be desirable to provide the art with a method to detect microplastics in water-based samples that is faster than the methods of the state-of-the-art, that avoids the generation of artifacts, and/or that allows the detection of small plastic microparticles in the range of 0.1 to 50 µm.

Any reference to prior art documents in this specification is not to be considered an admission that such prior art is widely known or forms part of the common general knowledge in the field.

The objective of the present invention is to improve or enrich the state of the art, and in particular to provide a method or a process to detect microplastics in water-based samples that is faster than the methods of the state-of-the-art, that avoids the generation of artefacts, and/or that allows the detection of small plastic microparticles with a size around 5 µm, or to at least provide a useful alternative.

The inventors were surprised to see that the object of the present invention could be achieved by the subject matter of the independent claim. The dependent claims further develop the idea of the present invention.

Accordingly, the present invention provides a process for detecting and characterizing plastic microparticles in a water-based sample comprising the analysis of the sample by spectral flow cytometry.

As used in this specification, the words “comprises”, “comprising”, and similar words, are not to be interpreted in an exclusive or exhaustive sense. In other words, they are intended to mean “including, but not limited to”.

As used in this specification, the words “particle” or “particles” are intended to describe a minute quantity or fragment of matter, for example, organic or inorganic matter or one or more micro-organisms, in a sample, for example a water-based sample.

The present inventors have shown that by using flow cytometry to detect microplastics in a water-based sample, they have found a rapid method to analyze, enumerate and identify microplastics in water-based samples as well as to distinguish them from organic matter, bacteria, humic acids, minerals and other materials.

No staining is needed, and - hence - the generation of artifacts through staining is avoided.

The inventors have further applied a dedicated machine learning algorithm for data analysis which allowed to recognize fingerprints of new types of materials based on the unique scattered light and fluorescence signal that is generated by different types of materials, so that new types of plastics, materials or contaminants in the samples can be detected as well.

Nile Red is sometimes used also in flow cytometry to stain lipids and cells, but it is not suitable for microplastics detection due to the lipophilic characteristic of the molecule that will bind to oil/fat droplets and organic materials or create micelles that may interfere with the measurements.

Fortunately, the inventors were surprised to see that flow cytometry could be used to detect microplastics in water-based samples without the need to use any staining.

Flow cytometry has been used in the past 30 years mainly for mammalian cells analyses, bacterial analyses and recently also for other submicron vesicles analyses (exosomes).

The method has several advantages compared to Nile Red staining and/or Raman or FTIR. Firstly, it dramatically increases the speed of analysis compared to spectroscopy methods: a sample can be processed quickly giving precise counts and rough estimation of particle sizes. Secondly, the Nile Red staining is not needed to detect microplastics in flow cytometry. Further, it avoids misleading artifacts; hence, the method provides a novel means for the identification of microplastics without relying on classical staining. Finally, the machine learning algorithm can be trained with new fingerprints to detect new types of plastics, materials or contaminants in the samples.

Hence, the present invention relates to the detection of plastic microparticles in a sample. In particular, the present invention relates to a process for detecting plastic microparticles in a water-based sample comprising the analysis of the sample by flow cytometry. The present invention also relates to a method for detecting plastic microparticles in a water-based sample comprising the analysis of the sample by flow cytometry.

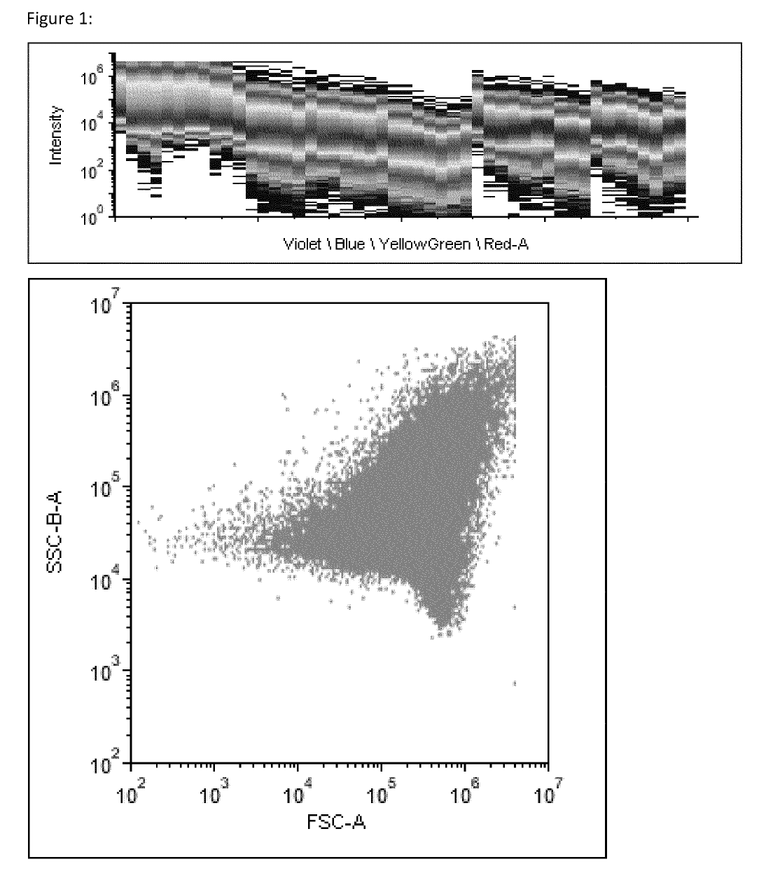

FIG. 1 shows an example of a PET spectrum and a scatter plot with particles bigger than 4 µm.

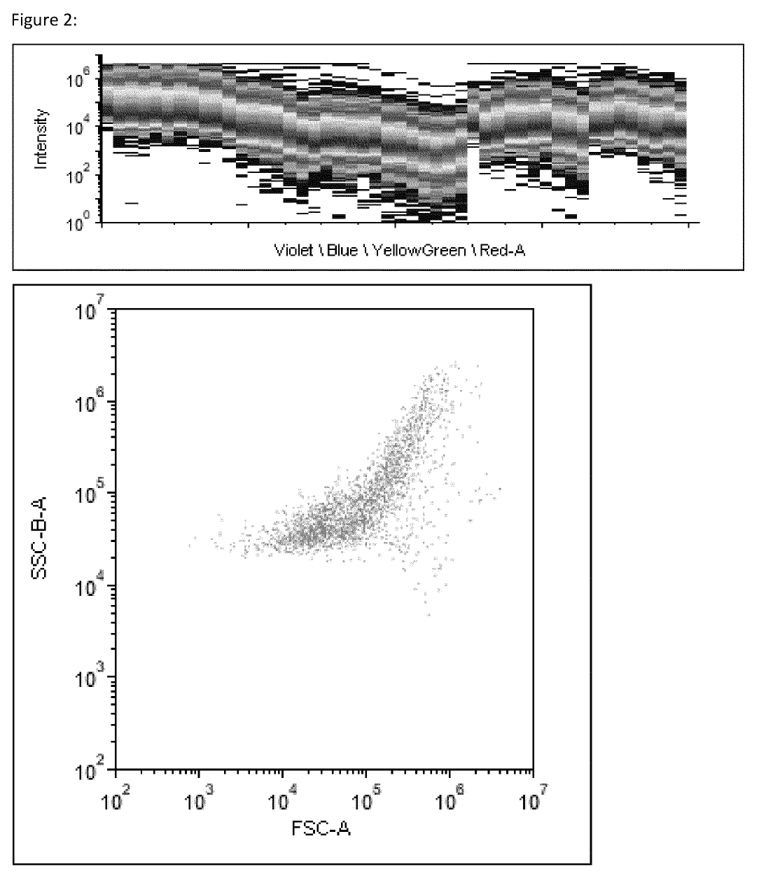

FIG. 2 shows an example of PE spectrum and scatter plot with particles bigger than 4 um.

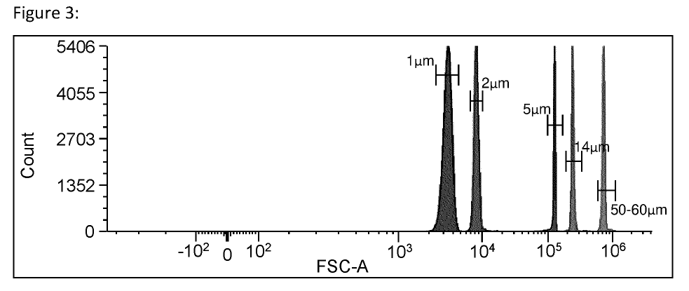

FIG. 3 shows sizing standards. The inventors have used a polystyrene particle size standard kit, flow cytometry grade, with standard particle sizes of 1 µm, 2 µm, 4 µm, 5 µm, and 50-60 µm.

Consequently, the present invention relates in part to a process or a method for detecting plastic microparticles in a water-based sample comprising the analysis of the sample by flow cytometry.

For the purpose of the present invention, plastic microparticles are small pieces of plastic with a size of less than 5 mm. This definition is in accordance with the proposal of the U.S. National Oceanic and Atmospheric Administration. For example, plastic microparticles may be small pieces of plastic with a length in the range of 1 µm - 5 mm. Because of the common use of 333 µm mesh neuston nets for capturing plankton and floating debris in water samples, plastic microparticles may also be small pieces of plastic with a size in the range of 333 µm - 5 mm.

For the purpose of the present invention, a water-based sample, shall be any sample with water as main component. For example, a sample shall be considered water-based, if it contains at least 90 vol-%, at least 95 vol-%, at least 96 vol-%, at least 97 vol-%, at least 98 vol-%, at least 99 vol-%, or at least 99.5 vol-% water. The water-based sample may be a sample of a food product. The term “food” shall mean in accordance with Codex Alimentarius any substance, whether processed, semi-processed or raw, which is intended for human consumption, and includes drink, chewing gum and any substance which has been used in the manufacture, preparation or treatment of “food” but does not include cosmetics or tobacco or substances used only as drugs. Further, for example, the water-based sample may be selected from the group consisting of water, for example drinking water such as still or sparkling water; tea; coffee; juice; lemonade; or fermented beverages, such as beer or wine for example. Further, for example, the water-based sample may be water.

Flow cytometry as a technology is known and is presently used for the analysis of biological cells. Flow cytometers are commercially available from several different suppliers such as Becton-Dickinson, Beckman-Coulter, BioRad, ThermoFisher, Cytek or Sony, for example.

Typically, flow cytometers comprise a flow cell, a measuring system, a detector, an amplification system, and a computer for analysis of the signals.

The principle of flow cytometry relies in a fluidic system that manages to pass single particles one after the other in front of one or multiple lasers. These lasers provide high intensity coherent light beams that illuminate the particles passing in the flow cell that can therefore scatter light and stimulate fluorescence light emission. For example, a series of dichroic mirrors and photomultipliers (PMTs) or avalanche photodiodes (APDs) detectors permit the detection and acquisition of emitted light associated to the particles passing in front of the lasers. The collection of emitted light results in a high number of variables for each particle in order to create a unique spectral fingerprint for each type of particle that can be subsequently categorized. The fingerprint of each particle is material-dependent. Consequently, these unique spectral fingerprints can be used to identify the particles, for example the plastic microparticles, in the sample.

Typical properties of the microplastic particles that can be detected with the process of the present invention include the following: type of plastic, particle size, for example as determined by a combination of FSC/SSC and time of flight, the specific autofluorescence signature and the abundance of a specific particle in the sample.

Analyzing the sample may comprise the collection of a water-based sample and passing it through a membrane. The membrane may be a nitrocellulose membrane or a silicon membrane, for example. Any particles collected by the membrane may then be washed from the membrane and the liquid sample may be subjected to laser light in a flow cytometer. Alternatively, the membrane may be dissolved by alkaline digestion and the obtained solution may be subjected to laser light in a flow cytometer.

Diffracted light and autofluorescence of the particles may be captured and quantified. As each particle type generates a unique spectrum, the resulting data can be used to analyze the microplastic content of the sample.

For example, the process of the present invention may comprise the following steps
transfer of a water-based sample containing particles through a flow cell, said flow cell being at least in part substantially transparent to at least one wavelength of interest,
irradiation of the water-based sample containing particles in the flow cell with light from at least one light source with one wavelength of interest,
recording of at least one optical property of said particles, resulting from the interaction of the particles with at least one light beam with a wavelength of interest in the flow cell,
characterization of said particles by using said recorded at least one optical property.

The functioning of a flow cytometer is well known to the skilled artisan and is described in the literature, for example in Critical Reviews in Biotechnology, 37:2, 163-176. The particles contained in the water-based sample may comprise microplastic particles. The water-based sample containing particles may be a natural water sample. In order to increase the concentration of the particles in the water-based sample, the particles in the water-based sample may also be concentrated. This may be achieved by any means known in the art, such as evaporation or filtration, for example. Filtration has the additional advantage that through the selection of the pore size in the filter or filters, the size range of the particles can be pre-selected. Consequently, in one embodiment of the present invention, the particles in the water- based sample are concentrated prior to the analysis.

The term “substantially transparent” shall mean for the purpose of the present invention at least 50%, at least 60%, at least 70%, at least 80%, at least 90%, at least 95%,or at least 99% transparent.

The light with one wavelength of interest is a light with a wavelength that allows recording of at least one optical property of said particles, resulting from the interaction of the particles with the light with the wavelength of interest. For example, the light source may have a wavelength in the range of visible light. A typical human eye will respond to wavelengths from about 380 to 740 nanometers.

The optical property resulting from the interaction of the particles with the light with the wavelength of interest can then be recorded. The recorded optical property of a particle in said sample can then be used to characterize the particle. Typically, particles produce unique optical properties, so that these can be used fingerprint-like to define certain properties of the particle. Such properties include the size and the nature of microplastic particles, for example.

Advantageously, the flow cytometry used in the process of the present invention is spectral flow cytometry. Spectral flow cytometry is well known in the art, and for example described in Biophotonics International, October 2004, p.36-40; Curr Protoc Cytom. 2013 Jan; CHAPTER: Unit 1.27; or Cytometry 95(8), August 2019, p.823-824.

Spectral flow cytometers improve the detection part compared to classical flow cytometers by using an array with a high number of filters and detectors that cover the whole visible spectrum from each laser emission to the near infrared (840 nm). The benefit of a spectral flow cytometer compared to a traditional one is that it enables the collection of a high number of variables for each particle in order to create a unique spectral fingerprint for each type of particle that can be subsequently categorized. The fingerprint of each particle is material-dependent and/or fluorophore-dependent.

The spectral flow cytometry may comprise a recording step which uses a detection array that covers at least 50%, at least 75% or at least 90% of the visible spectrum to record at least one optical property of said particles, resulting from the interaction of the particles with at least one light beam with a wavelength of interest in the flow cell. For example, the detection array may cover a wavelength range of about 380 to 560 nanometers, of about 560 to 740 nanometers, of about 400 to 600 nanometers, of about 380 to 640 nanometers, of about 390 to 730 nanometers, of about 480 to 740 nanometers, of about 380 to 720 nanometers, of about 400 to 740 nanometers, or of about 380 to 740 nanometers. In general, covering a large wavelength range has the advantage that the optical properties of a particle can be detected more completely. This in turn allows a more precise characterization of the particle.

The cytometer used for cytometry may be equipped with at least 1 laser. For example, it may be equipped with at least 2 lasers, at least 3 lasers, at least 4 lasers, at least 5 lasers, at least 6 lasers, at least 7 lasers, at least 8 lasers, at least 9 lasers, or at least 10 lasers. Using a larger number of lasers has the advantage that a larger number of optical properties of the particles can be detected at the same time resulting in a more complete “fingerprint”. For example, the cytometer used for cytometry in accordance with the present invention may be equipped with at least 4 lasers, for example 4 lasers with a wavelength of 405 nm, 488 nm, 561 nm, and 640 nm, respectively. The cytometer used for cytometry in accordance with the present invention may also be equipped with at least 5 lasers, for example 5 lasers with a wavelength of 355 nm, 405 nm, 488 nm, 561 nm, and 640 nm, respectively.

The at least one optical property of the particles, resulting from the interaction of the particles with at least one light beam with a wavelength of interest in the flow cell that is recorded in accordance with the present invention may be any optical property that allows a characterization of the particle. For example, diffracted light and/or fluorescence, such as autofluorescence, of the particles may be detected. If the particles are plastic microparticles, for example, during flow cytometry spectra comprising diffracted light and/or autofluorescence of the plastic microparticles may be recorded by at least one detector.

Any detector may be used that is suitable to detect the optical properties to be recorded. For example, an avalanche photodiode detector (APD) may be used, so that in one embodiment of the present invention, the diffracted light and autofluorescence of the plastic microparticles may be recorded by at least one avalanche photodiode detector (APD). Such APDs are readily commercially available from specialist suppliers, such as Hamamatsu, Osioptoelectronics, or Thorlabs, for example.

In accordance with the present invention, during cytometry, for example during flow cytometry fluorescence signals, forward scatter and/or side scatter and/or the other parameters are recorded by recording the height, the area and the width of the pulse signals. Recording the height, the area and the width of the pulse signals will further contribute to a refinement of the recorded fingerprints and will, hence, make the resulting detection and characterization of the plastic microparticles more precise.

Advantageously, the detection and characterization of the particles, for example the plastic microparticles, based on the recorded optical properties in accordance with the present invention is automated. Such an automation may be achieved by means of a computer implemented algorithm. For example, the recorded data may be processed by a machine learning algorithm that can distinguish and categorize each particle based on its unique spectrum to characterize said particles.

For example, the machine learning algorithm may collect and recognize the unique spectra of several different particle types that may be present in the water-based sample. These particle types may be different types of plastic, but may also be non-plastic particles. For example spectra from non-plastic particles may then be automatically substracted from the recorded optical properties of all the particles in the sample. The spectra of newly recognized non-plastic particles may be added to the list of collected spectra from non-plastic particles. This has the consequence that with time, more and more non-plastic particles can be automatically and reliably identified and excluded from the analysis of microplastic particles in the water-based sample. Hence, in one embodiment of the present invention, the recorded data are processed by a machine learning algorithm that involves the exclusion of spectra resulting from particles in the water-based sample which are not plastic-based.

Deep learning methods are machine learning methods based on multiple layers of artificial neuronal networks. Learning can be supervised, semi-supervised or unsupervised. Deep learning architectures have been applied in many fields today and have often produced results comparable to and in some cases superior to human experts.

For the purpose of the present invention a machine learning algorithm can be used that allows the identification and exclusion of spectra arising from non-plastic particles. For example, for the purposes of the present invention, the machine learning algorithm may comprise at last one algorithm selected from the group consisting of Deep Auto Encoder, Generative Adversarial Network, One Class Support Vector Machines, Isolation Forest, or combinations thereof.

The recorded data which comprise the recorded optical properties arising from at least a part of the particles in the water-based sample may be further processed by a machine learning algorithm that allows the categorization and/or identification of plastic particles from the sample. For example, this step may be carried out after exclusion of spectra resulting from particles in the water-based sample which are not plastic-based.

Hence, for example, the recorded data may be processed by a machine learning algorithm that involves the classification of plastic particles into predefined categories by using a supervised algorithm.

Such supervised algorithms are well known and describe the task of learning a function that maps an input to an output based on example input-output pairs. A supervised learning algorithm analyzes the training data and produces an inferred function, which can be used for mapping new examples (Stuart J. Russell, Peter Norvig (2010) Artificial Intelligence: A Modern Approach, Third Edition). Using a supervised algorithm has the advantage that the identification of particles will become more and more reliable and precise based on the quality and amount of data available in the training data.

The training data may comprise the unique combination of optical properties, for example, of the spectra of known reference particles. Such reference particles may comprise, for example, different types of plastic microparticles, but also other types of particles present in samples, such as micro-organisms or minerals, for example.

The predefined categories into which the analyzed particles, for example plastic microparticles, are classified into by processing the recorded data, for example with a supervised algorithm may include the following: Polyethylene, Poly(methyl methacrylate, Polystyrene, Polycarbonates, Polypropylene and Polyethylene terephthalate).

Several supervised algorithms are available that can be used to perform this classification. For example, the supervised algorithm may comprise at last one algorithm selected from the group consisting of Feed Forward Neuronal Network, Convolution Neuronal Networks, Random Forest, Support Vector Machines, Multilayer Perceptron, Logistic Regression, or combinations thereof.

The inventors have obtained particularly good results with the process of the present invention, if the particles in the water- based sample were concentrated prior to the analysis. For example, the particles in the water-based sample were concentrated by filtering an amount of the water-based sample to be tested through a nitrocellulose membrane with an average pore size in the range of 0.1-6 µm, digesting the membrane with a TBAH (tetrabutylammonium hydroxyde) 40% solution. After digesting the membrane the solution is diluted with LC-MS grade water.-The solution then contains the particles in a concentrated form. The solution was then analyzed by flow cytometry.

For example, the particles in the water-based sample were concentrated by filtering an amount of the water-based sample to be tested through a silicon membrane with an average pore size in the range of 0.1-6 µm, rinsing the membrane with LC-MS grade water and collecting the rinsing solution.-The solution then contains the particles in a concentrated form. The solution was then analyzed by flow cytometry.

Those skilled in the art will understand that they can freely combine all features of the present invention disclosed herein. In particular, features described for the product of the present invention may be combined with the use of the present invention and vice versa. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLES

### Method

Identification of microplastics in water or other beverages was carried out with a spectral flow cytometer equipped with 4 lasers (405 nm, 488 nm, 561 nm, 640 nm) or 5 lasers (355 nm, 405 nm, 488 nm, 561 nm, 640 nm). Diffracted light and autofluorescence of the microplastics were captured by a series of avalanche photodiodes (APDs) detectors in order to acquire each fluorescence signal, forward scatter and side scatter by recording the height, area and width of the pulse signals. Data collected were then processed through a machine learning algorithm that can distinguish and categorize each particle based on unique spectra.

Water samples (500 ml, 1 liter, 1.5 liters or 2 liters) were aseptically collected in a clean glass funnel and filtered with a stainless-steel manifold through a nitrocellulose membrane. The membrane was then digested with a solution of TBAH 40% in a clean glass tube. After digestion LC-MS grade water is added to dilute the solution and the content of the tube was analyzed with a spectral flow cytometer. The sample was acquired with a spectral flow cytometer with a FSC and SSC threshold set to avoid the detection of submicron particles. All parameters were recorded with height (H) and area (A) of the pulse. FSC, SSC and the first parameter of each array of detector for each laser was recorded with width (W) as well.

Sizing of the particles was determined by the FSC-A, SSC-A and V1-W parameters, when compared with known size particles (polystyrene particle size standard kit, flow cytometry grade).

Raw data were saved in FCS 3.0 format and imported in FCSExpress 7.0. The FCS files were analyzed with FCSExpress 7.0 and exported as CSV files with all the parameters for FSC, SSC and all the fluorescence detectors. The CSV files were further processed as described in the Data Analysis section below.

### Standard Preparation

All glassware have been rinsed with LC-MS Grade water (Pierce, cat number 51140), then washed in a SDS solution in LC-MS Grade water and put in an ultrasonic bath for 30 minutes. The glassware was then rinsed with Ethanol 70% and then rinsed again with LC-MS Grade water.

The plastic standards have been prepared by grinding different types of plastics. Each standard has been resuspended in a glass tube with a SDS solution to reduce buoyancy and acquired directly from the tube with a spectral flow cytometer as described above. At least 70000 particles were acquired for each standard. For the training of the algorithm less particles were used per standard.

### Data Analysis

The following pre-processing steps were performed on the flow cytometry data before applying the machine learning techniques:

Removal of additional exported variables: Features (independent variables) such as Time and FSC/SSC were removed from the dataset.

Data filtering: Particles for which at least one feature was out of scale or below the limit of quantification were removed from the analysis.

Outlier detection. Outlier particles were automatically detected and removed if they presented at least one value which was greater than 3 or less than -3 in z-score for the corresponding feature (different thresholds could be used to fine tune the stringency of outlier detection).

In addition, the data was transformed using a log transformation and scaled using a standard scale that standardize each row by removing the mean and scaling to unit variance.

Data exploration: Data was visualized using principal component analysis or with different dimensionality reduction tools to evaluate any cluster structure or the presence of global outliers.

### Classification

The water can contain an unknown number of different particles including plastic, bacteria, minerals, etc. While each particle type present in the water has a roughly defined spectrum, they have distinctly different types. Depending on the amount of negative particle types required to train the algorithm (types of non plastic particles present in the water) this problem can be addressed in two ways:

Closed Set Classifier: When the negative particles represent all types of particles present in the water to be classified that are not plastic (i.e. in a controlled environment where the water has always the same components).

Open Set Classifier: When an unknown number of different types of particles is present in the water to be classified (i.e. in a natural environment where the water can have an unknown number of components). The proposed deep learning process identifies and classifies the plastics from spectral flow cytometry data without requiring an exhaustive number of control particles (Open Set Classifier).

This process comprises two main steps:
I. Identification of plastic particles among other particles by using an unsupervised learning algorithm. In this first step, most of the negative particles (not plastic) and remaining outliers are excluded. As the number of plastics available for training has a roughly defined spectrum and the other types of particles are very sparse, to learn the intraclass correlations of each plastic standard a One Class Classifier is used. For example, an algorithm based on a deep auto-encoder that learns a lower dimensional representation of the data by trying to learn an approximation of the identity function. Two main parts compose an Autoencoder algorithm: the encoder that maps the input to a lower dimensional representation and a decoder that maps the lower dimensional representation to a reconstruction of the initial input. Other Algorithms, such as Generative Adversarial Networks, One Class Support Vector Machines or Isolation Forest can be used alternatively but their integration with the second part of this clas
II. Classification of plastic particles into the correct categories by using a supervised algorithm. In this second step, the detected plastic particles in the first step are classified according to each desired plastic type and/or color. The plastic classes must be defined beforehand and all the plastics for training the classifier must be correctly classified. It is considered that each plastic particle can belong to only one class of plastic. The classifier must also have a category that represents other types of particles that are not identified beforehand. To classify a plastic particle in one and only one plastic class, a multiclass classification algorithm is used. For example, a Deep learning model based on feed forward neuronal networks (i.e. Multi Layer Perceptron) will be able to evaluate the interclass correlations between all the defined plastic classes and choose the correct plastic class. In the case of using deep autoencoders for the first step, the pre-trained encoders can be integrated directly with a feed forward network. The feed forward net will be trained using as input the output of the encoder and will be able to not only categorize the plastics into the correct categories but also to exclude the particles that are not plastics with high accuracy. Other multi-class machine learning algorithms, such as Random Forest, Support Vector Machines, Multilayer Perceptron or Logistic Regression may be used alternatively. In the case of using one of those alternative algorithms, analysis is possible, but the performance will be impacted because the algorithm is trained without considering the existence of the one class classifier proposed in the first step.sification process will be optimal and performance issues are expected to occur. This type of algorithm is trained with the particles to be detected (plastic in this case) and using the recreation error as the objective parameter.

1. A process for detecting and characterizing plastic microparticles in a water-based sample comprising analyzing the sample by spectral flow cytometry.

2. The process in accordance with claim 1, wherein flow cytometry comprises the following steps
transfer of a water-based sample containing particles through a flow cell, the flow cell being at least in part substantially transparent to at least one wavelength of interest,
irradiation of the water-based sample containing particles in the flow cell with light from at least one coherent light source with one wavelength of interest,
recording of at least one optical property of the particles, resulting from the interaction of the particles with at least one light beam with a wavelength of interest in the flow cell, and
characterizing the particles by using the at least one recorded optical property.

3. The process in accordance with claim 1, wherein the spectral flow cytometry comprises a recording step which uses a detection array that covers at least 50% of the visible spectrum to record at least one optical property of the particles, resulting from the interaction of the particles with at least one light beam with a wavelength of interest in the flow cell.

4. The process in accordance with claim 1, wherein the cytometer used for cytometry is equipped with at least 4 lasers.

5. The process in accordance with claim 1, wherein during flow cytometry spectra comprising diffracted light and/or fluorescence of the plastic microparticles are recorded by at least one detector.

6. The process in accordance with claim 1, wherein diffracted light and autofluorescence of the plastic microparticles are recorded by at least one avalanche photodiode detector (APD).

7. The process in accordance with claim 1, wherein during flow cytometry fluorescence signals, forward scatter and/or side scatter are recorded by recording the height and area of the pulse signals.

8. The process in accordance with claim 1, wherein the recorded data are processed by a machine learning algorithm that can distinguish and categorize each particle based on its unique spectrum to characterize the particles.

9. The process in accordance with claim 8, wherein the recorded data are processed by a machine learning algorithm that involves the exclusion of spectra resulting from particles in the sample which are not plastic-based.

10. The process in accordance with claim 8, wherein the machine learning algorithm comprises at last one algorithm selected from the group consisting of Deep Autoencoder, One-Class Classification, Generative Adversarial Network, One Class Support Vector Machines, Isolation Forest, and combinations thereof.

11. The process in accordance with claim 8, wherein the recorded data are processed by a machine learning algorithm that involves the classification of plastic particles into predefined categories by using a supervised algorithm.

12. The process in accordance with claim 8, wherein the supervised algorithm comprises at last one algorithm selected from the group consisting of Feed Forward Neuronal Network, Convolution Neuronal Networks, Random Forest, Support Vector Machines, Multilayer Perceptron, Logistic Regression, and combinations thereof.

13. The process in accordance with claim 1, wherein the particles in the water- based sample are concentrated prior to the analysis.

14. The process in accordance with claim 1, wherein the particles in the water-based sample are concentrated by filtering an amount of the water-based sample to be tested through a nitrocellulose membrane with an average pore size in the range of 0.1-6 µm, digesting the nitrocellulose membrane in a alkaline solution comprising 40% TBAH in water, and diluting the obtained solution.
